(19) United States
(12) Patent Application Publication
(10) Pub. No.: US 2007/0269852 A1
(43) Pub. Date: Nov. 22, 2007

(54) Method and apparatus for detecting airborne pathogens using quantum dots

(76) Inventor: James Johan Muys, Auckland (NZ)

Correspondence Address: JAMES J. MUYS, 27B DELL AVENUE, REMUERA, AUCKLAND, 1005, omitted

(21) Appl. No.: 11/435,993

(22) Filed: May 18, 2006

(51) Int. Cl. C12Q 1/04; C12M 1/34

(52) U.S. Cl. 435/34; 977/902; 435/287.1

(57) ABSTRACT

A method has been invented to detect and identify airborne pathogens and their properties based on the affinity of water-soluble quantum dot bioconjugates. A plurality of different sized quantum dot bioconjugates, where each size is conjugated with a different functional biological material or biomaterial, are combined in solution to form an assay. The airborne pathogens are then dissolved in the solution, where they contact the quantum dot bioconjugates. If the quantum dot bioconjugates have an affinity to the dissolved pathogens they will bind together. The quantum dots bound to the pathogens are then separated from those that remain unbound by filtration. An activation light source is then used to excite the quantum dots present in the filtered solution. A detector then records the fluorescence emitted from each size of the quantum dot bioconjugates—The properties of the airborne pathogens can then be deduced based on the known affinities of the functional material conjugated to those quantum dot bioconjugates, which are absent, as well as present in the filtered solution.

### CROSS-REFERENCE TO RELATED APPLICATIONS

Not Applicable

### FEDERALLY SPONSORED RESEARCH

Not Applicable

### BACKGROUND OF THE INVENTION

1. Field of the Invention

The field of the invention relates to the biological applications of a composition comprising of water-soluble quantum dots. More specifically, this invention includes a method and apparatus for applying functional quantum dot bioconjugates to identify airborne pathogens.

2. Prior Art

Airborne pathogens are extremely hazardous due to their high contamination, fatal toxicity and containment difficulty. Traditional analytical tools such as optical microscopy are largely ineffective due to the nanoscale dimensions of pathogens (typically<5 μm) and current devices designed to detect pathogens are often limited to the detection of a specific pathogen and are not easily scalable to different pathogenic types.

A widely used method to identify pathogens is fluorescence, which involves tagging a fluorescent dye or marker to pathogens based on a chemical or biological binding affinity of the functional material attached to the dye marker, known to those skilled in the art. Upon binding of the dye to the target biological material, an activation light is used to excite the dye into fluorescence. Traditionally, organic dye fluorophores have been the favored material for fluorescent applications. However, they have numerous limitations when used to tag biological materials.

Most of the limitations with traditional organic dyes are a result of the extremely limited absorption and emissive capabilities. The first shortcoming is that the peak emission wavelength of organic dyes cannot be altered—Each dye corresponds to a different molecule with a given emission wavelength, or fluorescent color, that is determined by nature. Therefore, applications that make use of light frequencies that do not correspond to the emission peaks of pre-existing organic dyes cannot be performed. The second shortcoming is the narrow absorption pattern of organic dyes—Dyes tend to display absorption peaks that are not always in convenient regions of the spectrum, making the excitation of various organic dyes costly and challenging.

The third shortcoming is that of uneven absorption and emission peaks—Organic dyes have a tendency to produce “shoulders” in the geometry of their emission and absorption peaks, which is a major disadvantage in applications that require Gaussian type emission patterns for predictability and reliability. An additional shortcoming is that of stability—The lifetime of organic dyes varies but is generally low relative to that of other tagging mechanisms, and organic dye fluorescence is almost controlled entirely by the molecular bonding properties of each individual dye.

Finally, incident radiation absorbed by an organic dye molecule moves electrons into excited states, whereupon they decay and release light radiation. This emission cannot be altered because it corresponds to pre-set excited states of the dye molecule that are inherent to every molecule of that type. Whereas the light emission ranges and possible forms of organic dyes are very limited, quantum dots, another fluorescent tag can be made to emit light at any wavelength in the visible and infrared ranges and can be inserted almost anywhere, including in liquid solutions, dyes, paints, epoxies, and sol-gels.

Quantum dots are semiconductor fluorescent nanoscale light-emitting semiconductor crystals, which are spherical in shape and have superior fluorescent properties to organic dyes. When an activated light is applied, the quantum dots discretely fluoresce. Several terms are often used interchangeably to refer to the emission of light by quantum dots following light stimulation, they are; emission, luminescence, photo-luminescence and fluorescence. Similarly, several terms used to describe the state of the quantum dot when it fluoresces; these are excited, photo-excited, and activated.

Quantum dots are generally synthesized with type III-V (e.g. InAs and InP) or type II-VI (e.g. CdS, CdSe, CdTe, and ZnSe) column elements from the periodic table. Often additional capping shells, layers, or molecules are added to the underlying semiconductor nanocrystal in order modify their physical properties, such as for surface functionalization (Chan et al., 2002, Curr. Opin. Biotech. 13:40-46) or to render their naturally hydrophobic nature hydrophillic for water-solubility.

Integration of quantum dots in biology was achieved in breakthroughs showing that highly luminescent quantum dots could be made water-soluble and subsequently biocompatible using surface modification techniques such as silica/siloxane coatings (Gerions, 2001, J. Phys. Chem. B 105:8861-8871; and Bruchez et al., 1998, Science 281:2012-2015) or by direct absorbtion of bifunctional ligands (Chan et al., 1998, Science 281:2016-2018). With properties superior to traditional fluorescent proteins and organic dyes, quantum dots are showing promise as the new biological label (Watson et al., 2003, Biotechniq. 34:296-300; Michalet et al., 2005, Science 307(5709):538-544; and Chan et al., 2002, Biotechnol. 13:40-46).

Quantum dots can be rendered “functional” by linking biological molecules such as proteins (Mattoussi et al., 2000, J. Am. Chem. Soc. 122:12142-12150), DNA (Mitchell et al., 1999, J. Am. Chem. Soc. 121:8122-8123), peptides (Whaley et al., 2000, Nature 405:665-668) and nucleic acids (Niemeyer, 2001, Angew. Chem. Int. Ed. 40:4128-4158), using bioconjugative techniques known by those skilled in the art.

Methods for covalently linking biological molecules to create functional quantum dots or “bioconjugates” (Goldman et al., 2002, J. Am. Chem. Soc. 124:6378-6382; Jaiswal et al., 2004, Nature Methods 1:1; Mattoussi et al., 2000, J. Am. Chem. Soc. 122:12142-12150; and U.S. Pat. No. 6,114,038 to Castro (2000); U.S. Pat. No. 6,855,551 (2005) to Bawendi et al.; and U.S. Pat. No. 6,468,808 to Nie (2002)), can be achieved using a variety of chemistries and cross-linking molecules to attach a primary functional biological material, known to those skilled in the art. Typically, methods are based on the hydrophilic attachment of the primary functional biological material or biomaterial to the water soluble quantum dots directly or indirectly. If the biomolecules are attached indirectly, linker molecules are often used to form intermediary bonds between the quantum dot and biological molecules.

Bioconjugation methodologies and techniques can be classified into five general mechanisms (Chan et al., 2002, Curr. Op. Biotech. 13:40-46): (1) Electrostatic attraction (Mattoussi et al., 2000, J. Am. Chem. Soc. 122:12142-12150); (2) Biofunctional linkages (Chan et al., 1998, Science 282:2016-2018); (3) Silanization (Bruchez et al., 1998, Science 281:2013-2015); (4) Hydrophobic attraction; and (5) Nanobead linkages (Han et al., 2001, Nat. Biotech. 19:631-635).

Bioconjugation methods for attaching antibodies to quantum dots are based on well-known chemistries such as the coupling of thiols to maleimide groups using intermediary reactive groups such as primary amines, alcohols, and carboxylic acids. In cases where the functional biological material such as the antibody cannot be directly conjugated to the quantum dots, an intermediary, or linker molecule, such as biotin, actin or protein-IgG, known to those skilled in the art, can be used. A typical example of methods employing bioconjugative techniques is by modifying the underlying carboxyl groups present on the quantum dots, or by optimizing the surface loading of amino groups to increase conjugation efficiency and specificity. A further example is peptide modification of the quantum dots through the amino or carboxyl terminus groups. For examples of other bioconjugate techniques used to attach different functional materials to quantum dots refer to; Bioconjugate Techniques (Academic Press, New York (1996)).

Labeling of pathogens has been demonstrated using quantum dots (Hahn M A et al., 2005, Anal. Chem. 1;77(15):4861-9; Agrawal et al., 2005, J. Virol. 79(13):8625-8; Lee et al., 2004, Appl. Environ. Microbiol. 70(10):5732-6; Zhao, 2005, Clin. Lab. Sci. 18(4):254-62; Agrawal et al., 2006, Anal. Chem. 15;78(4):1061-70; Bentzen et al., 2005, Nano Lett. 5(4):591-5; and Malamud et al., 2005, Adv. Dent. Res. 18(1):12-6). However, at present there remains no effective methodology or apparatus for simultaneously applying several differently functionalized quantum dot bioconjugates for detecting airborne pathogens.

3. Objects and Advantages

It is an object of this invention to enable the rapid detection of airborne pathogens by the quantitative binding of quantum dot bioconjugates. The advantages of using quantum dots over traditional organic fluorophore dyes and fluorescent proteins are realized in their superior optical and spectral properties. Traditional organic dyes suffer from several problems, such as photobleaching, spectral cross-talking and narrow excitation—Quantum dots have the potentional to overcome these problems (Xiaohu, 2003, Tren. Biotech. 21:9). Quantum dots are broadly compared with organic dyes as being superior with respect to (Alivisatos, 1996, Science 271:933-937); composition and size dependent tunable emission wavelengths; large absorption cross sections; wide absorption profiles; good photostability; and narrow emission spectra.

Firstly, quantum dots possess superior fluorescent properties, which are directly tunable through modification of the size of the underlying semiconductor crystal. Additionally, quantum dots have a centered, symmetric, bell-shaped and narrow bandwidth less than 30 nm, which provides a neat and predictable emission spectrum. Excitation of quantum dots is easy due to their broad absorbance range, resulting in a high quantum yield and descretely detectable luminescent emission peaks. The broadband absorption properties of quantum dots make simultaneous activation advantageous in systems employing several sized quantum dots.

Moreover, the Stokes shift means that in the visible spectral region there is a shift of 15 nm between the emission and absorption wavelengths. This large Stokes shift allows the quantum dot emission signals to be separated and easily distinguishable from the background fluorescence, a task not done easily with conventional dyes (Yang et al., 2000, Proc. Natl. Acad. Sci. U.S.A. 97:1206-1211). Furthermore, quantum dot activation can be achieved using light sources shorter than the emission wavelengths of the quantum dots and thus made effectively independent from excitation source. In addition, quantum dots have an incredible height intensity and brightness allowing much longer integration times, photostability and lifetime characteristics.

Accordingly, from the superior advantages of using quantum dots over traditional dyes, it is an object of this invention to combine quantum dots with airborne pathogens in an apparatus that provides a convenient and easily adaptable method for pathogenic detection and analysis. This goes some way to overcoming the above disadvantages, or which at least provides a useful choice over existing approaches for detection of airborne pathogens.

### SUMMARY

The present invention provides a method and apparatus for detecting airborne pathogens using water-soluble quantum dots bioconjugated with different functional biological materials and biomaterials. In this invention the quantum dot bioconjugates are designed to simultaneously test for different airborne pathogens. Comprised in this invention is a filtration method for trapping the airborne pathogens dissolved in a mixed solution consisting of functional quantum dot bioconjugates and non-functional quantum dots. A light activation source is used to excite the quantum dots and a detector is used to record the distinguishing fluorescent wavelengths emitted from each of the different sizes of quantum dots present in the solution. Moreover, the quantum dot bioconjugates in the solution will be conjugated with functional materials allowing for the simultaneous detection and analysis of airborne pathogens.

### DRAWINGS—FIGURES

FIG. 1A illustrates the fluorescent intensity spectrum of the solution comprised of quantum dots before contact with the airborne pathogens.

FIG. 1B illustrates the fluorescent intensity spectrum of the solution comprised of quantum dots after contact with the airborne pathogens and subsequent filtration.

FIG. 2A illustrates the key components and apparatus used to dissolve the airborne pathogens in the solution of quantum dots.

FIG. 2B depicts the method of dissolving airborne pathogens into the solution using vacuum suction.

FIG. 2C illustrates the binding of the quantum dot bioconjugates with the dissolved pathogens.

FIG. 2D shows the filtration method used to separate the pathogens from the solution.

FIG. 2E depicts the apparatus used to activate and detect the quantum dots in the solution.

DRAWINGS - Reference Numerals

| | | | |
|---|---|---|---|
| 110 | test tube | 112 | solution |
| 114 | 7 nm quantum dot bioconjugates | 116 | 5 nm quantum dot |
| | | | bioconjugates |
| 118 | 6 nm non-functional quantum dots | 120 | stopper |
| 122 | inlet tube | 124 | outlet tube |
| 126 | flexible tube | 128 | vacuum-pressure pump |
| 130 | airborne anthrax spores | 132 | filtration tube |
| 134 | fluid filter | 136 | lamp |
| 138 | detector | 140 | vacuum inlet |

### DETAILED DESCRIPTION

The term “airborne pathogens” as used herein are micro-organisms capable of causing diseases, particularly in humans, which are spread by droplets expelled into the air, typically through coughing or sneezing. Included in this definition are allergenic, toxigenic, respiratory and non-respiratory pathogens, such as mold, fungi, bacteria, spores, viruses, and actinomycetes. Examples of pathogens and diseases capable of forming in the air are severe acute respiratory syndrome (SARS), avian influenza A bird flu virus (e.g. H5N1 strain), anthrax and ricin.

The term “bioconjugate” as used herein refers to methodologies and techniques used to attach functional biological materials and biomaterials such as antibodies to the quantum dots, which can be done using any stable physical or chemical associations known by those skilled in the art.

The term “functional” as used herein refers to the quantum dot being bioconjugated with biomaterials or biological materials.

The term “functional materials” as used herein refers to biomaterials and biological materials.

The term “non-functional” as used herein refers the quantum dots not being modified with functional materials intended to bind to the airborne pathogens dissolved in the solution. More specifically, non-functional quantum dots are used to distinguish from those functional quantum dot bioconjugates present in the solution, which have been modified and designed to bind or attach to pathogens.

The present invention provides a method and apparatus to quantitatively deduce the properties of airborne pathogens using quantum dot bioconjugates. The present invention is premised on the advantageous discovery a solution comprising of a plurality of different sized water-soluble quantum dot bioconjugates can be functionalized and used as an in vitro assay, revealing information on the characteristics of airborne pathogens by dissolving them in the solution. The present invention is used to determine the properties of the airborne pathogens based on a biological or chemical attraction of the functional material conjugated to the quantum dots, known to those skilled in the art. The present invention is also premised on the discovery that the quantum dot bioconjugates can conveniently be brought in contact and made to interact with the airborne pathogens by dissolving the airborne pathogens in solution. The present invention is further premised on the discovery that quantum dot bioconjugates having no attraction to the dissolved pathogens can be separated from those that do by filtration.

In view of the above, the present invention provides in one embodiment a plurality of functional, water-soluble quantum dot bioconjugates. It is an object of this invention to use quantum dot bioconjugates of different sizes—By functionalizing quantum dots of a given size and known peak fluorescence wavelength, multiple sized quantum dots of different functionality can be combined in a single system.

Accordingly, a preferred embodiment of the present invention provides a water-based solution comprising of a plurality of different sizes of water-soluble quantum dots functionalized using bioconjugation techniques, known by those skilled in the art. The assortment of functional quantum dots combined in the solution is designed to interact with airborne pathogenic material of a specific type or class—Where at least one of the quantum dot sizes present in the solution is non-functional or not bioconjugated with any biological materials or biomaterials.

Hence by combining different quantum dot bioconjugates in the solution it is useful for testing, analysis and diagnosis. The sizes of the quantum dots used in the solution are variable (typically between 1-12 nm), such that each size has a peak emission wavelength, which is easily distinguishable from the fluorescent emissions of the adjacent sized quantum dots present in the solution. Furthermore, in order to avoid large cross-over interference it will be advantageous to use quantum dots emitting within a narrow and predictable band range. This enables more quantum dots of different sizes to be included in the solution. As most biological systems are present in aqueous solutions, it is a preferred embodiment of this invention to use water-soluble quantum dots.

Another embodiment of the inventive methods includes contacting the quantum dot bioconjugates with the airborne pathogens in the solution and allowing them to interact, and bind to each other if a biological or chemical affinity exists. Quantum dot bioconjugates can be conjugated with functional materials using bioconjugation techniques based on the detection of a specific type of airborne pathogen. Hence, if the presence of a specific type of airborne pathogen is to be detected then the quantum dots will be functionalized with materials known to have an affinity specifically to those pathogens. That way the presence, or at least absence, of those airborne pathogens can be verified by the binding of quantum dot bioconjugates conjugated with functional materials with known properties.

Accordingly, one embodiment of the inventive methods comprises of dissolving and mixing the airborne particles in the solution. To achieve this, airborne pathogens are dissolved into the solution by pumping air, in which the airborne pathogens are formed, into the solution using a vacuum-pressure pump. The vacuum-pressure pump can be operated such that the vacuum input on the pump sucks the air through the solution or the pressure output on the pump forces the air into the solution. The amount of air pumped into the solution will depend on the quantity of pathogenic material present. Typically, air will be pumped into the solution continually for a period lasting a minute up to an hour, depending on the vacuum-pressure pump specifications. If an affinity exists between the functional material on the quantum dot bioconjugates and the dissolved airborne pathogens, binding will occur and the quantum dot bioconjugates will tag the dissolved airborne pathogens.

In another embodiment, the present invention provides a method for separating the solution into two separate components using filtration—(1) airborne pathogens dissolved in the solution and the bound quantum dot bioconjugates, and (2) unbound quantum dot bioconjugates and non-functional quantum dots formed in the filtered solution. After a period of time lasting several seconds up to an hour after dissolving the airborne pathogens into the solution, the solution is then filtered. The unbound quantum dots pass though due to the small size of the quantum dots relative to the pore or mesh size of the fluid filter. The dissolved pathogens and the attached quantum dots will trap in the fluid filter resulting in a filtered solution consisting only of unbound quantum dots.

Fluid filters are generally porous materials used to remove particulates and other solid and semi-solid contaminants from fluid or solution. They are rated with respect to the diameter of the largest spherical particle that will pass through the openings, referred to as pores or mesh, in the filter. Hence the pore or mesh size and shape determine the maximum pathogen size that can pass through the fluid filter.

Filter elements can be surface or depth filters—Surface filters are made of closely woven fabric or treated paper with a uniform pore or mesh size. Fluid flows through the pores of the fluid filter material and contaminants are stopped on the filter's surface. This type of element is designed to prevent the passage of a high percentage of solids of a specific size.

On the other hand, depth filters are composed of many fabric or fiber layers, which provide many paths for the solution to flow through. The pores must be larger than the rated size of the filter if particles are to be retained in the depth of the media rather than on the surface. Filter elements may consist of woven mesh, micronic, porous metal, or magnetic type. Often filter mediums are composed of organic and inorganic fibers integrally bonded by epoxy resin and faced with a metallic mesh upstream and downstream for protection and added mechanical strength. The filter can use layers of very fine stainless-steel fibers drawn into a random but controlled matrix, wire mesh, felts or papers, perforated sheets or wound strands. Metal mesh or porous filters can be inter-bonded to prevent movement of fibers or particles relative to one another, thus assuring absolute pore size control. Porous material construction determines the maximum differential pressure which can be imposed upon the medium. Cores and other reinforcements and backup materials, known to those skilled in the art, can be placed against the filter to enhance the ability to withstand greater pressures.

The fluid filter will be able to pass quantum dots but yet be able to capture pathogens and any attached quantum dots. Hence, the mesh or pore sizes in the fluid filter will typically be greater than 20 nm but less than 5 um, depending on the pathogenic material to be captured. Examples of materials that can be used as the fluid filter include; strainers, nylon wool, gauze, mesh, mesh baskets, gauze, membrane filters, porous membranes, nano-patterned fabricated membranes (e.g. Silicon nitride membranes with lithography patterned holes) known to those skilled in the art, or any mesh or passive filtration system capable of the fine filtration and separation of particles in fluid. Examples of porous membrane filters include isopore polycarbonate membranes and mixed cellulose ester membranes, which are commercially available in different pore sizes from Millipore Corporation, U.S.A.

The small size (typically<5 μm) of airborne pathogens requires fine filtration of the solution. Fluid filters capable of filtering fine particles often use pore filters such as membranes, rather than mesh filters such as strainers, which are typically used to filter macro-molecules like tissues or cells. While pore filters will almost offer finer filtration, they require additional forces other than gravity for filtration. In most instances the filter will require the solution to be assisted through using forces such as vacuum suction, pressure pumping or centrifugation. Hence, it is an inventive method to effectively filtrate the airborne pathogens dissolved in the solution using external pressure-vacuum pumps, or centrifuge devices, which create vacuum, centrifugal or pressure forces to assist in filtration. Examples are portable vacuum-pressure pumps such as high output and chemical duty pumps—available from Millipore Corporation, U.S.A and centrifuges (catalogue numbers 5418 and 5424)—available from Eppendorf International Limited.

In cases where there is a large quantity of pathogens dissolved in the solution, large solution volume (i.e. >1 ml), or where the solution contains different sizes or types of pathogenic material, it is a property of this invention to use several fluid filters, or different types of filters for filtration. The properties of the filter will depend on the airborne pathogens dissolved in the solution such as their size and the quantity. Hence, if the airborne pathogens are viral spores 200 nm in diameter, then the mesh of pore size of the filter must be at least half that diameter for effective filtration. Another embodiment of the inventive methods, which will become apparent from the illustrations, includes use of the filter in an apparatus designed to effectively implement the inventive methods. The filter will be mounted in a tube such as a test tube, enabling filtration while reducing contamination and spillage of the solution.

It is an object of this invention to employ an activation source capable of applying an excitation light to the quantum dot bioconjugates and non-functional quantum dots present in the filtered solution. The presence of quantum dots is detectable upon exposure to a light, or lamp activation source designed to excite, or activate the quantum dots to emitting a fluorescent peak (also known as luminescent peak). Light emissions from the quantum dots will form several distinct wavelength bands, determined by the number of quantum dot sizes present in the solution. Examples of possible activation sources that can be used to excite the quantum dots to fluoresce are helium/neon lasers, green lasers, argon lasers and light emitting diodes (LEDs). Examples of lamps that can be used as the activation source in the invention are ultraviolet (U.V), deuterium, tungsten, sodium, halogen, mercury, and xenon lamps. Examples of light emitting diodes (LEDs) include U.V-LEDs and deep U.V-LEDs. Several lamps, LEDs and light sources can be used simultaneously and their intensity and frequency modulated and controlled using slits, irises and filters.

Detection of quantum dots is achieved electrically using photosensitive detectors such as spectrometer devices. The advantage of electrical detection is in miniaturization, systems integration and automation. Accordingly, the photosensitive detector used in this invention will be able to effectively and simultaneously detect the different radiation wavelengths emitted from the different sized quantum dots. Spectrometers such as spectrophotometers, fluorescent spectrometers and fluorometers are packaged photosensitive devices capable of sensing small amounts of radiation in a wavelength range. Typically the different sizes of quantum dots emit radiation in the deep-U.V to the near infrared (n-IR) light wavelengths (200-1100 nm).

The advantages of using spectrophotometers are they often use multi-elemental silicon photodiodes—These are semiconductor devices capable of detecting extremely minute quantities of light radiation in the form of photons at different wavelengths. This enables the simultaneous measurement of a source emitting at several different wavelengths. Ideally the detector will closely match the radiation bands emitted by the variable sized quantum dots. Examples of photo-detectors that can be used as the detector in the invention include charge-coupled device (CCD) sensors, such as Hamamatsu S9840, Sony ILX511 and Toshiba TCD 1304AP sensors.

It is an object of the invention to employ optoelectronic or semiconductor accessories capable of detecting and amplifying the impinging radiation to the detector. Additional spectrometer components like photomultipliers, optical filters, focusing lenses, beam splitter devices, optical fibers, mirrors and filter gratings with varying slit widths, and collimators, known to those skilled in the art can be used to deflect, direct or split the emitted light onto the detector.

Due to the light intensities emitted from the quantum dots being relatively low, it is an object of this invention to enhance and focus their emissions onto the detector for more accurate sensing. Hence, techniques such as employing fiber optical cables can be used to focus fluorescent emissions from the quantum dots precisely onto the sensing area on the detector. In order to establish whether quantum dots are indeed present based on signals produced from the detector, it is also an object to incorporate additional detectors positioned at various locations. Such an example would be positioning detectors at locations prime for detecting light from the quantum dots bound to biological materials, which have been captured in the filter, as well as placing detectors primed to detect the presence of quantum dots in the filtered solution.

It is another object of the invention to determine and record the relative fluorescent intensities produced in the solution prior to contact with the airborne pathogens as well as after filtration. This enables calibration of the respective fluorescent intensities of the quantum dots present and can be used to deduce the changes in the fluorescent intensities in the solution before and filtration. Hence, if the concentration of one size of quantum dot present in the solution is higher than the others, the fluorescent intensity peak at its emitting fluorescent wavelength will also be higher. Note, also, that the fluorescent intensities are dependent on the size of the quantum dots. Hence, a given concentration of quantum dots of a smaller size will have lower fluorescent peak intensity than those of a larger size.

In the case of a significant imbalance in the quantum dot concentrations it is a method of the invention to adjust the balances by increasing the quantum dot concentrations until the fluorescent intensity peaks are balanced to within a stated variation or standard deviation degree—This degree will depend on the number of different sized quantum dots incorporated in the solution, but typically would be less than 3% relative deviation for a solution comprised of 5 different sized quantum dots.

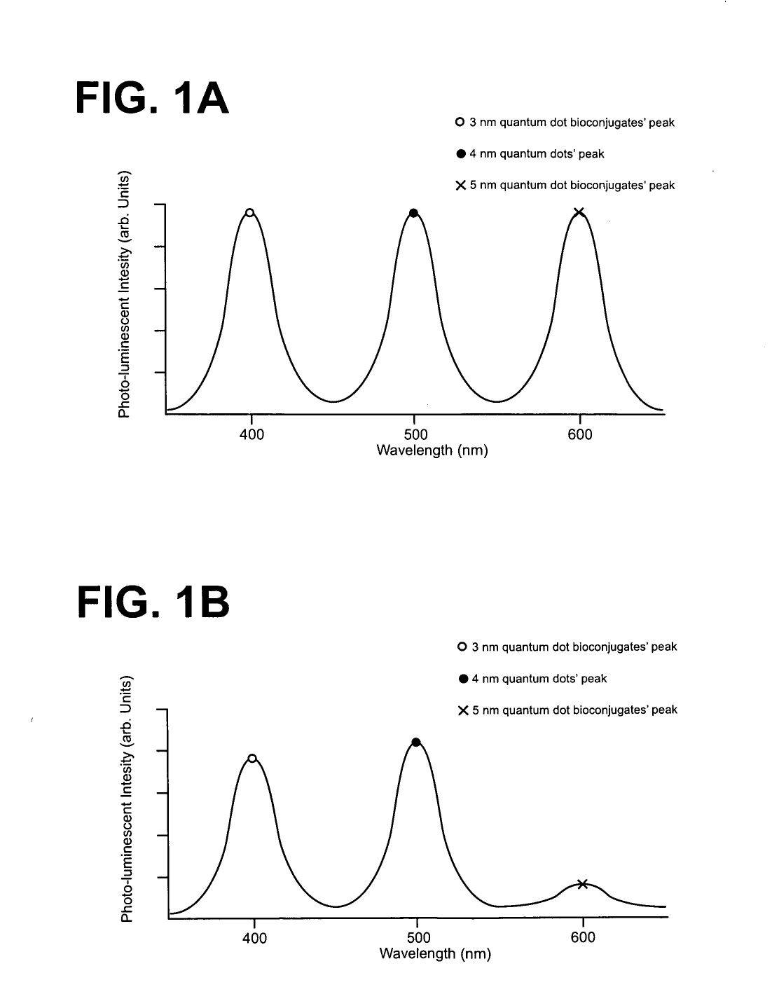

As shown in FIG. 1A, a photo-luminescent intensity (arbitrary units) spectra depicts the three fluorescent peaks emitted from a solution comprised of plurality of 3 and 5 nm differently functional quantum dot bioconjugates and a plurality of 4 nm non-functional quantum dots, respectively. The intensities of the fluorescent emissions have been balanced by adjusting the concentrations of the different sized of quantum dots until the respective fluorescent peaks are level as illustrated in the spectra.

FIG. 1B shows the photo-luminescent intensity spectra from the solution after the airborne pathogens are dissolved in the solution and the solution is filtered, according to the methods described in the invention. A diminished intensity is seen at the 5 nm quantum dot bioconjugates' peak fluorescence wavelength—Both relative to the other quantum dots peaks, as well as with respect to that in FIG. 1A. Thus it can be said that the 5 nm quantum dot bioconjugates and the airborne pathogens have an affinity to each other.

Hence, information about the pathogens can be deduced by correlating the quantum dot bioconjugates absent from the filtered solution by their conjugated functional material. Furthermore, the non-functional quantum dots used in the solution provide information on whether methods of the invention have been performed correctly and their presence in the filtered solution provides experimental validation. The fluorescent intensity of the non-functional quantum dots also provides an additional calibration for calculating the relative intensities of the quantum dot bioconjugates—There will inevitably be small quantities of all sizes of quantum dots present, information about the degree of interaction and binding with other quantum dots can be gathered by relative comparison of their intensities.

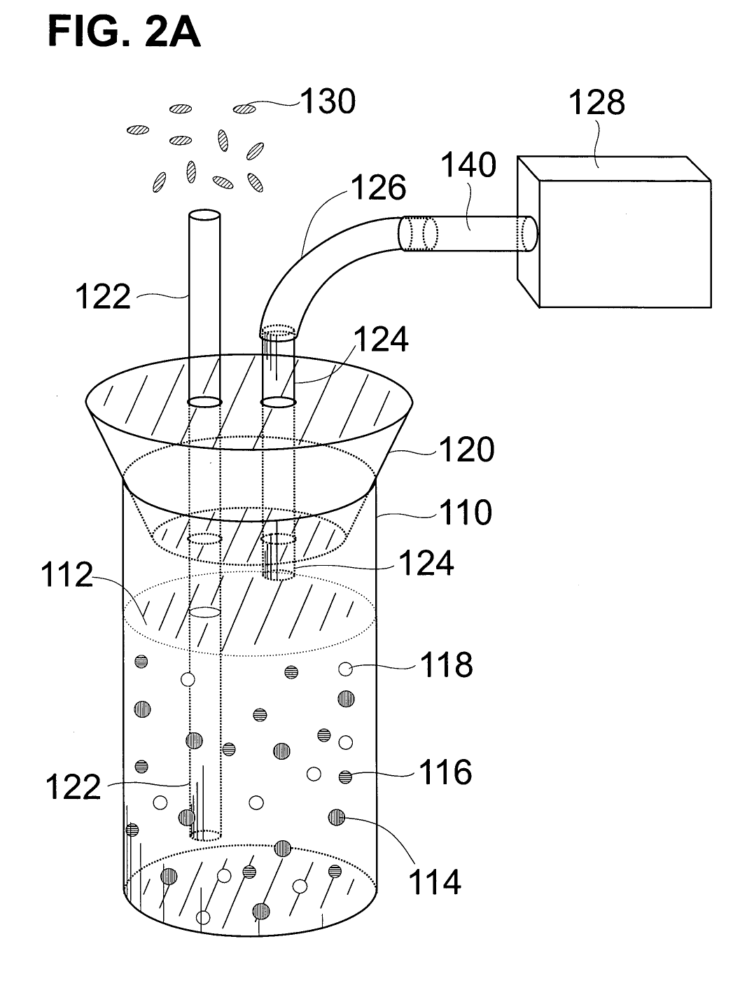

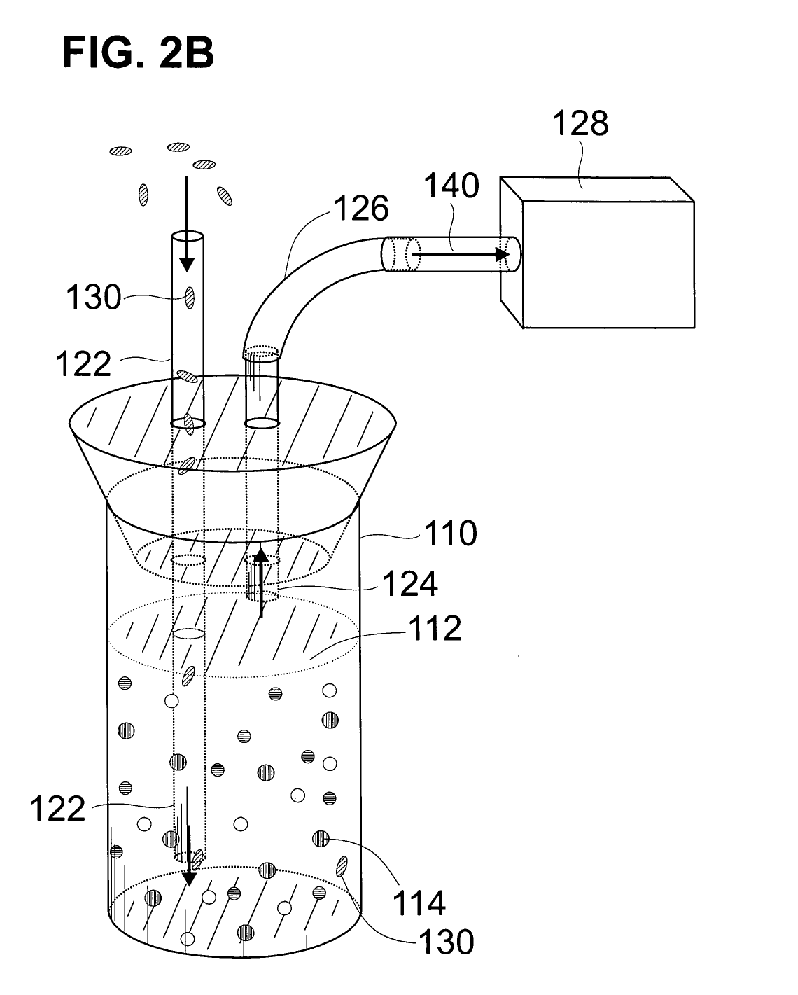

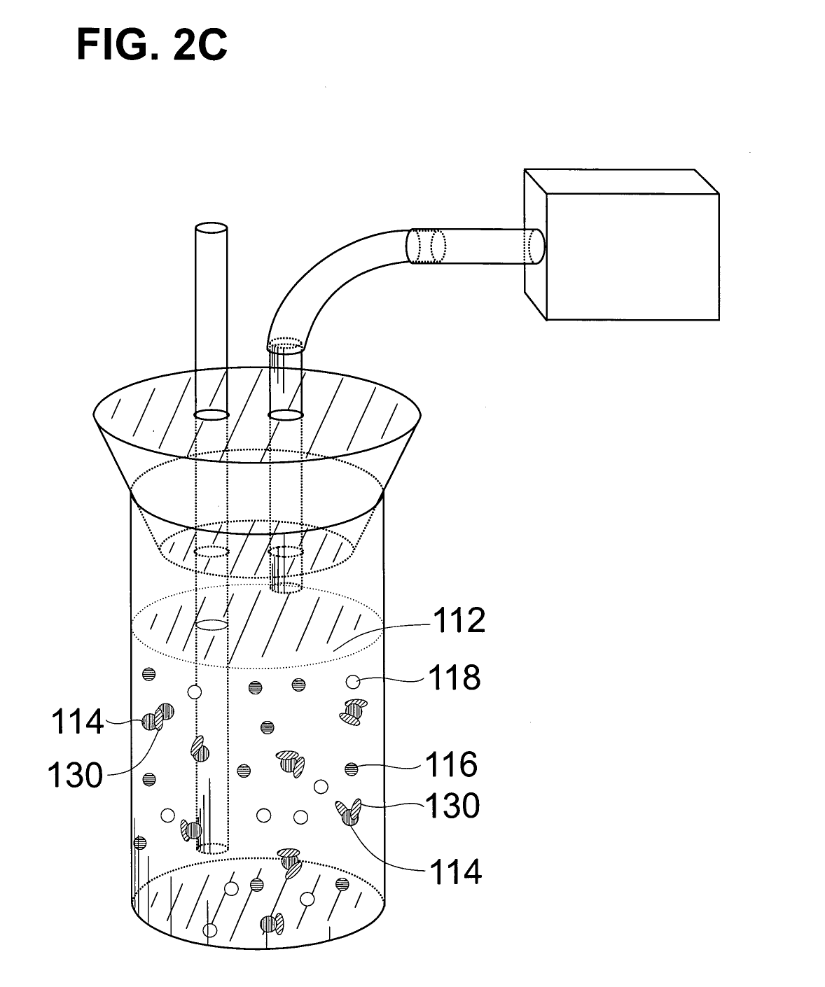

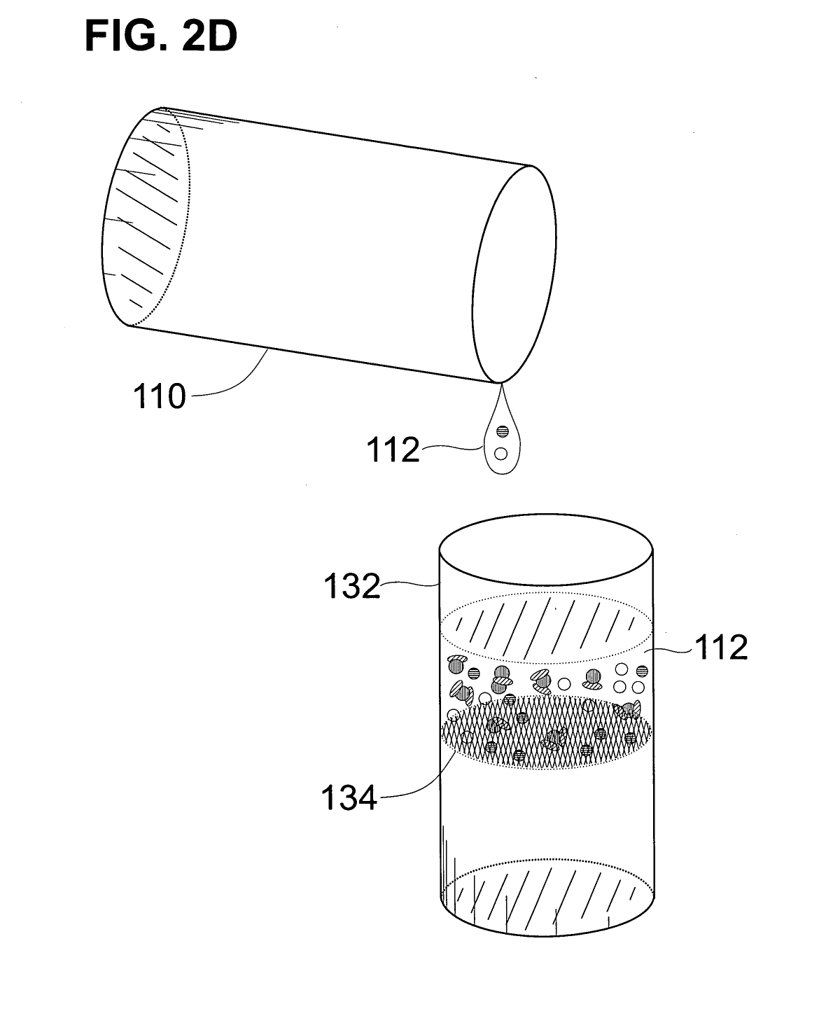

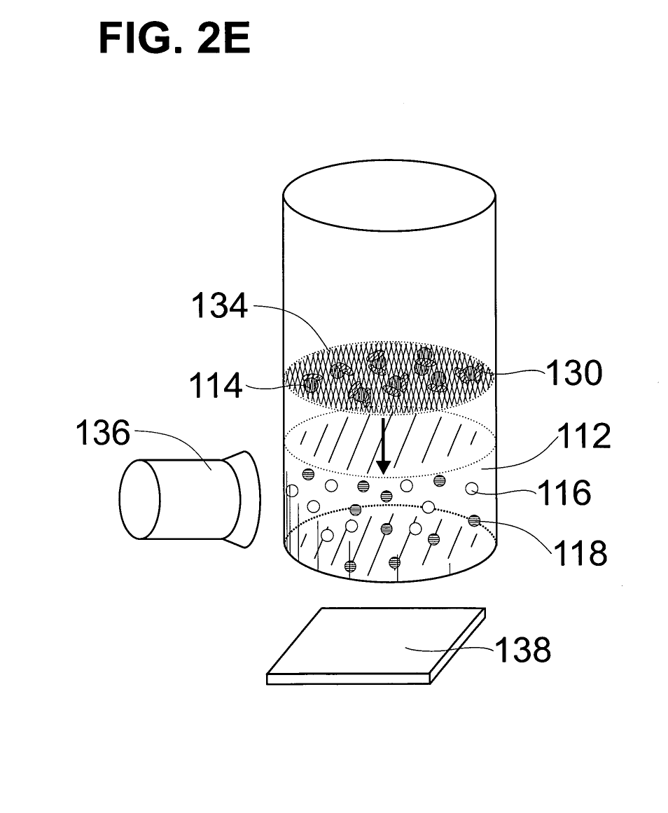

Methods of the invention are now illustrated in the steps shown in FIGS. 2A to 2E. To begin with, FIG. 2A depicts the key components of the apparatus used to perform the inventive methods—A test tube 110 containing a solution 112 consisting of equal concentrations of; a plurality of 7 nm quantum dot bioconjugates 114 conjugated with a functional material; a plurality of 5 nm quantum dot bioconjugates 118 conjugated with a different functional material; and a plurality of 6 nm non-functional quantum dots 116. The open end of test tube 110 is sealed by a stopper 120, which contains two holes to allow an inlet tube 122 and an outlet tube 124 into test tube 110. Inlet tube 122 extends to submerse in solution 112, whereas outlet tube 124 is not submersed in solution 112. Outlet tube 124 is connected to a flexible tube 126, which is connected to a vacuum inlet 140 on a vacuum-pressure pump 128. Outside of test tube 110 are a plurality of airborne anthrax spores 130 formed in the air.

As shown in FIG. 2B, operation of the apparatus and the first step of the inventive methods begins by dissolving airborne anthrax spores 130 in solution 112, by turning vacuum-pressure pump 128 ‘on’, which creates suction in flexible tube 126 and causes air from within test tube 110 to be sucked out through outlet tube 124, forcing airborne anthrax spores 130 formed in the air from the outside and into solution 112.

As shown in FIG. 2C, in the next step, airborne anthrax spores 130, which have been dissolved into solution 112 interact with the quantum dots. In this instance binding occurs between airborne anthrax spores 130 and 7 nm quantum dot bioconjugates 114 due to a positive affinity with the functional material on 7 nm quantum dot bioconjugates 114. Whereas there is no affinity, and hence no binding, with 6 nm non-functional quantum 118 dots nor 5 nm quantum dot bioconjugates 116.

As shown in FIG. 2D, another inventive method includes filtering solution 112 to separate airborne anthrax spores 130 and any bound quantum dots. After a period lasting several minutes to an hour, solution 112 is poured from test tube 110 into a filtration tube 132, containing a fluid filter 134 with pores 100 nm wide.

As shown in FIG. 1E, solution 112 passes through fluid filter 134 and airborne anthrax spores 130 and the attached 7 nm quantum dot bioconjugates 114 are captured in fluid filter 134. Whereas 6 nm non-functional quantum dots 118 and 5 nm quantum dot bioconjugates 116 pass through due to pores size in fluid filter 134 being much larger than that of the quantum dots. After filtration, 6 nm non-functional quantum dots 118 and 5 nm quantum dot bioconjugates 116 are activated by a lamp 136, which is focused on solution 112. Their fluorescent emissions are then detected by a detector 138 and their peak fluorescent intensities are identified and compared relative to each other. Hence, it can be said that airborne anthrax spores 130 have an affinity to 7 nm quantum dot bioconjugates 114, due to their absence from solution 112 post-filtration. In addition, it can also be said that airborne anthrax spores 130 do not have an affinity to 5 nm quantum dot bioconjugates 116. It is expected that 6 nm non-functional quantum dots 118 are present in solution 112 post-filtration as they are purposely added to act as a non-binding control and their absence would indicate an inconclusive test result. Thus properties, or at least inferences, about those airborne anthrax spores 130 can be made from the known properties of the functional materials conjugated to 7 nm quantum dot bioconjugates 114 and 5 nm quantum dot bioconjugates 116, and whether they bound to the airborne anthrax spores 130 dissolved in solution 112 or not.

While the above description contains much specificity, these should not be construed as limiting the scope of the invention, but as exemplifications of the presently preferred embodiments thereof. Many other ramifications and variations are possible within the teachings of the invention. For example, the test tube can have other shapes; the filter can be placed or orientated at different positions or angles in the test tube; the detector can be placed at different angles with respect to the lamp, etc.

Thus the scope of the invention should be determined by the appended claims and their legal equivalents, and not be the examples given.

I claim:

1. An apparatus for quantitatively detecting a plurality of airborne pathogens and their properties, comprising:
(a) a solution consisting of a plurality of quantum dot bioconjugates and a plurality of non-functional quantum dots,
(b) a vacuum-pressure pump for pumping said airborne pathogens into said solution,
(c) a fluid filter for filtration of said solution,
(d) a light source for activating said quantum dot bioconjugates and said non-functional quantum dots,
(e) a detector for detecting fluorescent emissions of said quantum dot bioconjugates and said non-functional quantum dots,
whereby said airborne pathogens are contacted with said quantum dot bioconjugates by dissolving said airborne pathogens into said solution by means of forcing air in which said airborne pathogens are formed into said solution using said vacuum-pressure pump, said quantum dot bioconjugates with an affinity to said airborne pathogens bind together, said quantum dot bioconjugates with no affinity remain unbound, said non-functional quantum dots also remain unbound, wherein said airborne pathogens and the bound quantum dot bioconjugates are separated from said solution by means of filtration of said solution using said fluid filter, by which said light source then activates said quantum dot bioconjugates and said non-functional quantum dots in the filtered solution and said detector detects their fluorescent emissions.

2. The apparatus of claim 1 wherein said fluid filter traps said airborne pathogens dissolved in said solution during filtration.

3. The apparatus of claim 1 wherein said fluid filter is mounted in a tube.

4. The apparatus of claim 1 further comprising a pump for suction of said solution through said fluid filter.

5. The apparatus of claim 1 further comprising a centrifuge for assisting in the filtration of said solution through said fluid filter.

6. The apparatus of claim 1 wherein after filtration of said solution, said quantum dot bioconjugates bound to said airborne pathogens are excited by said light source and their fluorescent emissions detected by said detector.

7. The apparatus of claim 1 wherein said quantum dot bioconjugates and said non-functional quantum dots are of a plurality of sizes, where each size fluoresces at a peak wavelength different from the other said sizes.

8. The apparatus of claim 7 wherein concentrations of different said sizes of said quantum dot bioconjugates and said non-functional quantum dots in said solution are balanced such that their peak wavelength intensities are the same.

9. The apparatus of claim 7 wherein each size of said quantum dot bioconjugates is conjugated with a functional material different from the other said sizes.

10. The apparatus of claim 1 wherein fluorescent emissions of the quantum dots in said solution are measured before contact with said airborne pathogens.

11. A method for separating a solution consisting of a plurality of quantum dot bioconjugates of different sizes conjugated with a plurality of functional materials, from a plurality of airborne pathogens by filtration, comprising the steps of:
(a) contacting said quantum dot bioconjugates with said airborne pathogens in said solution by pumping air in which said airborne pathogens are formed into said solution, wherein said quantum dot bioconjugates with an affinity to said airborne pathogens bind together, and wherein said quantum dot bioconjugates with no affinity to said airborne pathogens remain unbound,
(b) filtering said solution using a fluid filter, wherein said fluid filter filters said airborne pathogens and the bound quantum dot bioconjugates, and wherein the unbound quantum dot bioconjugates pass through,
(c) activating the unbound quantum dot bioconjugates contained in said solution to emit a plurality of fluorescent peaks using an activation source,
(d) detecting said fluorescent peaks emitted from the unbound quantum dot bioconjugates using a detector and determining the bound quantum dots by the absence or reduction in their said fluorescent peaks,
whereby the properties of said airborne pathogens can be deduced by the affinities of said functional materials on said quantum dot bioconjugates and whether they were bound to said airborne pathogens or not.

12. The method of claim 11 wherein each size of said quantum dot bioconjugates is conjugated with said functional materials different from the other said sizes.

13. The method of claim 11 wherein a plurality of non-functional quantum dots of different sizes to said quantum dot bioconjugates comprise said solution.

14. The method of claim 13 wherein said non-functional quantum dots are of different sizes to said quantum dot bioconjugates and emit different said fluorescent peaks.

15. A method for detecting the presence of an airborne pathogen using a solution consisting of a plurality of quantum dot bioconjugates of a plurality of sizes and a plurality of non-functional quantum dots, comprising the steps of:
(a) applying a light source designed to activate the quantum dots in said solution and detecting a plurality of fluorescent peaks produced from the different sized quantum dots using a detector,
(b) balancing the intensities of said fluorescent peaks by changing the concentrations of the quantum dot sizes in said solution, such that said fluorescent peaks are level with respect to each other to within a known deviation,
(c) dissolving said airborne pathogen into said solution by pumping air in which airborne pathogen is carried into said solution using a pressure-vacuum pump,
(d) contacting said quantum dot bioconjugates with said airborne pathogens in said solution, wherein said quantum dot bioconjugates with an affinity to said airborne pathogens will attach with said airborne pathogens, and wherein said non-functional quantum dots and said quantum dots with no affinity to said airborne pathogens remain unattached,
(e) separating said quantum dot bioconjugates attached to said airborne pathogens from the unattached quantum dots by filtration of said solution through a fluid filter, wherein said airborne pathogens and the attached quantum dot bioconjugates will trap in said fluid filter,
(f) activating the quantum dots in the filtered solution by applying said light source and recording their said fluorescent peaks using said detector,
whereby said fluorescent peaks of said quantum dot bioconjugates and said non-functional quantum dots in the filtered solution can be used to indicate the properties of said airborne pathogens from the binding interaction of said quantum dot bioconjugates.

16. The method of claim 15 wherein said non-functional quantum dots are used as a control to determine the relative fluorescences of said quantum dot bioconjugates.

17. The method of claim 15 wherein post filtration said quantum dot bioconjugates attached to said airborne pathogens in said fluid filter are activated with said light source and said fluorescent peaks detected using said detector.

18. The method of claim 15 wherein said vacuum-pressure pump is used to assist filtration of said solution through said fluid filter.

19. The method of claim 15 wherein said quantum dot bioconjugates of said sizes are conjugated with different functional materials to one another.

20. The method of claim 15 wherein said quantum dot bioconjugates and said non-functional quantum dots of said sizes fluoresce at different said fluorescent peaks not imposed by one another.
